(19) United States
(12) Patent Application Publication
(10) Pub. No.: US 2009/0029911 A1
(43) Pub. Date: Jan. 29, 2009

(54) Liquid Human Growth Hormone Formulation Containing Polyethylene Glycol

(76) Inventors: Ashley Martin Williams, Winnipeg (CA); Terrance Jimmy Sereda, Winnipeg (CA)

Correspondence Address: Ade & Company Inc;PO Box 28006, 1795 Henderson Highway, Winnipeg, MB, R2G 0P1, CA

(21) Appl. No.: 10/573,571

(22) PCT Filed: Sep. 27, 2004
(86) PCT No.: PCT/CA04/01698

§ 371 Date: Mar. 22, 2007

Related U.S. Application Data
(60) Provisional application No. 60/505,432, filed on Sep. 25, 2003.

(51) Int. Cl. A61K 38/18; A61P 5/06

(52) U.S. Cl. 514/12

(57) ABSTRACT

A stable pharmaceutically acceptable aqueous formulation containing human growth hormone, a buffer, polyethylene glycol, a tonicifier such as a sugar alcohol, and optionally, an antimicrobial agent and optionally, a chelating agent. Also disclosed are associated means and methods for preparing, storing and using such formulations.

### PRIOR APPLICATION INFORMATION

This application claims priority under 35 USC § 119(e) to Provisional Patent Application Ser. No. 60/505,432 filed on Sep. 25, 2003.

### FIELD OF THE INVENTION

The present invention is directed to pharmaceutical formulations or compositions containing human growth hormone (hGH) and to methods for making and using such formulations. More particularly, this invention relates to such pharmaceutical formulations having increased stability during long term storage in aqueous formulation.

### BACKGROUND OF THE INVENTION

Human growth hormone is a hormone used for treatment of hypopituitary dwarfism even though it has been proposed to be effective in the treatment of burns, wound healing, dystrophy, bone knitting, diffuse gastric bleeding and pseudarthrosis, and other conditions for which it is effective. See, for example, U.S. Pat. No. 4,342,832. The major biological effect of hGH is to promote growth. The organ systems affected include the skeleton, connective tissue, muscles, and viscera such as liver, intestine, and kidneys. Growth hormone exerts its action through interaction with specific receptors on cell membranes. Advances in recombinant DNA technology have allowed human growth hormone to be produced from heterologous sources including bacterial cells.

Recombinant proteins are preferentially combined into pharmaceutical formulations that retard protein degradation. There are two basic mechanisms that are responsible for the degradation of proteins, i.e., chemical and physical. Chemical degradation refers to modifications to the protein that involve events such as deamidation, oxidation or disulfide interchange. Physical degradation refers to changes to the overall structure of the protein, i.e., events such as denaturation (i.e., tertiary structure unfolding).

As proteins tend to undergo degradation in an aqueous environment, lyophilized human growth hormone formulations were developed to slow the rate of degradation, examples of which are described in the background sections of U.S. Pat. No. 5,763,394. However, such lyophilized formulations are subject to reconstitution errors, thereby decreasing dosing accuracy, as well as complicating the use of the product clinically, which may decrease patient compliance.

Liquid hGH formulations would solve the above noted limitations of lyophilized formulations. However, long-term storage of hGH in liquid formulations is subject to physical degradation resulting from the interaction of hGH with interfaces such as air/water and vial/water, which may result in denaturation of the hGH protein. Upon denaturation, hydrophobic surfaces may become exposed which may promote the interaction and unfolding of additional protein which can then cause the formation of protein aggregates. These aggregates may then desorb from the surface and cause the formation of precipitates. These types of interactions may occur during handling due to agitation or at a slow rate during long-term storage of the hGH formulation.

As described in U.S. Pat. No. 5,763,394, non-ionic surfactants can be added to hGH formulations to prevent protein loss due to aggregation and results in a liquid pharmaceutical formulation that is stable during long-term storage. It is generally accepted that there are two mechanisms by which non-ionic surfactants stabilize liquid protein formulations against aggregation: surfactant binding to a surface on the protein and surfactant binding to interfaces which compete with the protein. One example of a non-ionic surfactant stabilizing proteins by the first mechanism is Tween, which has been shown can bind either to the surface of the native hGH molecule or to a folding intermediate of hGH. In either case, surfactant binding is based on a specific surfactant to protein stoichiometry, and thus the concentration of surfactant required in a formulation is dependent on the protein concentration. Non-ionic surfactants that stabilize proteins via the second mechanism are independent of protein-surfactant stoichiometry, but provide protection against aggregation at concentrations above the critical micelle concentration (CMC) of the surfactant. Such non-ionic surfactants adsorb to the air-liquid interface and compete with the protein for such interface. This inhibits denaturation of the proteins due to protein adsorption.

PEG is not a non-ionic surfactant as defined in U.S. Pat. No. 5,763,394 (“the '394 patent”):

“ . . . include a polysorbate, such as polysorbate 20 or 80, etc., and the poloxamers, such as poloxamer 184 or 188, Pluronic™, polyols, and other ethylene/polypropylene block polymers, etc. Amounts effective to provide a stable, aqueous formulation will be used, . . . ”

The '394 patent describes surfactants that are amphiphilic molecules with water soluble head groups and hydrophobic tails, or polymers composed of hydrophobic and hydrophilic subunits. A typical example of a surfactant that fits this model is Tween, in which PEG forms the hydrophilic head and a C₁₁ or C₁₇ alkyl chain forms the hydrophobic tail. PEG is not an amphiphilic molecule in that it does not have a water soluble head group and a hydrophobic tail. PEG is not a Poloxamer, which denotes a symmetrical block copolymer, consisting of a core of PPG (Polypropylene glycol) that is polyoxyethylated to both its terminal hydroxyl groups, i.e. conforming to the general type (PEG)ₓ-(PPG)_{y}-(PEG)ₓ.

PEG is a homopolymer of ethylene-oxide monomer units, and thus PEG is not a polymer composed of hydrophobic and hydrophilic subunits. Additionally, PEG is not a polyol, which are typically defined as non-reducing sugars, sugar alcohols, sugar acids, lactose, pentaerythritol, water-soluble dextrans, and Ficoll. Thus, PEG is not a molecule described or contemplated in the '394 patent as a non-ionic surfactant.

PEG does not Function as a Non-Ionic Surfactant

Previous studies have demonstrated that PEG does not provide protection from agitation-induced aggregation when used at the same mole ratios as the surfactant Tween (Bam, NB, et al, J Pharm Sci. 1998 December;87(12):1554-9). Thus, this demonstrates PEG is not protecting proteins from aggregation by binding to proteins in the same manner as the non-ionic surfactant Tween protects proteins.

Although PEG is known to lower the surface tension of water, it is not considered a surfactant as defined in the '394 patent. Many agents, such as proteins, lower the surface tension of water but are not considered surfactants. PEG is classed as a solubilizing agent (i.e., an agent which will increase the solubility of a substance which has low inherent solubility) and the Handbook of Pharmaceutical Excipients indicates that the functional category of PEG is that of an ointment base, plasticizer, solvent, suppository base and tablet and capsule lubricant (Pharmaceutical Press 2^{nd} Edition, 1994, p. 355).

Preferential exclusion is a phenomenon wherein a protein solution and a co-solvent (i.e., any component that comprises a significant portion of the solution) do not interact favorably and the co-solvent is preferentially excluded from the surface of such protein. The result is a sphere of hydration forming around the protein. Although PEG is known as a preferential exclusion co-solvent, the interaction of PEG and a protein is dependent on the chemical nature of the protein surface. Thus, it is difficult to predict the effect of PEG on any given protein. For some proteins, PEG interacts with the proteins, lowering the melting temperatures (Lee, L L -Y and Lee, J C, Biochemistry, 1987 (26): 7813-9). Such lowering of a protein's Tₘ may have a “destabilizing” effect. For example, in an aqueous solution of 1% PEG-8000 and storage at 4° C., the activity of LDH decreased 10% in 8 days (Mi, Y, et al, PDA J Pharm Sci Technol. 2002 May-June;56(3):115-23). Without PEG, the activity of LDH decreased 30% over the same time period, suggesting PEG provides only minimal protection to LDH during long term storage. In comparison, U.S. Pat. No. 5,763,394 teaches that the presence of non-ionic surfactant results in less than 1% aggregation of hGH after 18 months of storage.

Due primarily to the preferential exclusion mechanism, PEG is well known as a potent precipitation agent for proteins and is used extensively in the field of X-ray crystallography because of its strong precipitant properties. The solubility of a protein can decrease by 95% or more in the presence of PEG4000 and concentrations from approximately 5 to 35% (Atha D. H. and Ingham K. C., J Biol Chem. 1981 Dec. 10;256(23):12108-17). Consequently, use of PEG as a preferential exclusion co-solvent in protein solutions is typically associated with protein precipitation.

Formulations of growth hormone (GH) with polyethylene glycol (PEG) are known in the art. However, all such GH formulation with PEG teach high concentrations of PEG molecules that form a gel after injections and result in a slow release of GH. For example, U.S. Pat. No. 4,041,155 describes a long-acting growth hormone release-inhibiting composition suitable for subcutaneous or intramuscular injection comprising GH and about 80% polyethylene glycol 400, at which concentration hGH is precipitated.

U.S. Pat. No. 6,011,011 teaches a sustained release formulation of GH comprising nonaqueous compositions of polyethylene glycol such as PEG 300 to PEG 600 dissolved in glyceryl triacetate or triacetin.

Such sustained release formulations are typically viscous and thus require a large bore needle for injection. An hGH formulation for human application would ideally be administered through a small bore needle to minimize discomfort to the patient. Additionally, such sustained release formulations are typically used in veterinary or agricultural settings to increase the growth rate of bovines and porcines; however, use in humans requires a more controlled administration.

PEG is also used as a binder for the formulation of pellets or tables for parenteral sustained release of GH, as described in U.S. Pat. No. 4,917,685. There is no teaching that the PEG itself can stabilize the GH. In fact, U.S. Pat. No. 4,917,685 teaches that stabilizers are highly polar molecules such as sugars, amino acids, polymers of amino acids and choline salts.

It is an object of the present invention to provide an aqueous hGH pharmaceutical formulation stabilized by PEG that demonstrates physical and chemical degradation comparable to aqueous hGH formulations stabilized with Tween or Poloxamer.

It is an object of the present invention to provide an aqueous pharmaceutical hGH formulation that is long term storage stable and does not contain a non-ionic surfactant as defined in the '394 patent. It is a further objective of this present invention to provide a liquid pharmaceutical hGH formulation wherein the hGH is stabilized by preferential exclusion of a co-solvent.

### SUMMARY OF THE INVENTION

One aspect of the invention is an aqueous human growth hormone formulation comprising human growth hormone, a buffer, a tonicifying agent and effective amount of Polyethylene glycol, and optionally an antimicrobial agent in a sterile pharmaceutically acceptable liquid.

A further aspect of the invention is a method of inhibiting physical and chemical degradation of a human growth hormone in an aqueous formulation during storage comprising mixing a human growth hormone and a polyethylene glycol in the range 5 mg/mL to 50 mg/mL.

According to a first aspect of the invention, there is provided a human growth hormone formulation comprising:

a) 1 mg/ml to 20 mg/ml human growth hormone,

b) buffer system providing pH 5.5 to pH 7,

c) a tonicifying agent, and

d) an effective amount of Polyethylene glycol,

in a sterile pharmaceutically acceptable liquid.

According to a second aspect of the invention, there is provided a method for using human growth hormone comprising the steps of

A) formulating said human growth hormone into an aqueous liquid formulation comprising:

a) 1 mg/ml to 20 mg/ml human growth hormone,

b) buffer system providing pH 5.5 to pH 7,

c) 5 mg/mL to 50 mg/mL polyethylene glycol, and

d) a tonicifying agent,

in a pharmaceutically acceptable, injectable sterile aqueous vehicle,

B) storing said formulation as an aqueous liquid for from six to 18 months at 2° C. to 8° C. thereby forming a stored formulation; and

C) directly injecting said stored formulation into a patient in need of human growth hormone therapy.

According to a third aspect of the invention, there is provided a method for using human growth hormone comprising the steps of

A) formulating said human growth hormone into an aqueous liquid formulation consisting essentially of:

a) 1 mg/ml to 20 mg/ml human growth hormone,

b) buffer system providing pH 5.5 to pH 7,

c) 5 mg/mL to 50 mg/mL polyethylene glycol,

d) 20 to 100 mg/mL of a tonicifying agent and

e) an antimicrobial agent,

in a pharmaceutically acceptable, injectable sterile aqueous vehicle,

B) storing said formulation as an aqueous liquid for from six to 18 months at 2° C. to 8° C. thereby forming a stored formulation; and

C) directly injecting said stored formulation into a patient in need of human growth hormone therapy.

According to a fourth aspect of the invention, there is provided a method of making a storage stable aqueous formulation of human growth hormone comprising mixing said human growth hormone into an aqueous, pharmaceutically acceptable vehicle which includes

a) 1 mg/ml to 20 mg/ml of said human growth hormone;

b) buffer providing pH 5.5 to pH 7;

c) 5 mg/mL to 50 mg/mL polyethylene glycol; and

d) 20 to 100 mg/mL of a tonicifying agent;

wherein said aqueous, pharmaceutically acceptable vehicle and is capable of storage for 6 to 18 months at 2 to 8° C.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

### A. Definition

The term “human growth hormone” or “hGH” is used to denote human growth hormone produced by methods including natural source extraction and purification, and by recombinant cell culture systems. Its sequence and characteristics are set forth, for example, in Pearlman and Bewly (Pharm Biotechnol. 1993;5:1-58). The terms likewise cover biologically active human growth hormone equivalents, e.g., differing in one or more amino acid(s) in the overall sequence. Furthermore, the terms used in this application are intended to cover substitution, deletion and insertion amino acid variants of hGH, or posttranslational modifications. Two species of note are the 191 amino acid native species (somatropin) and the 192 amino acid N-terminal methionine (met) species (somatrem) commonly obtained recombinantly.

The term “long term stable”, as it relates to a pharmaceutical liquid hGH formulation, is a formulation that results in less than 3 to 4% aggregation of hGH and less than 15% deamidation of hGH when such pharmaceutical liquid hGH formulation is stored at 2° C. to 8° C. In one embodiment, such liquid hGH formulation is long term stable for at least 24 months, and in an alternate embodiment, such liquid hGH formulation is long term stable for 6 to 18 months, and in an alternate embodiment, such formulation is stable at least 12 months. As will be obvious to one skilled in the art, the long term stability of hGH may be determined directly by incubating the formulations for the above noted times, or the long term stability may be predicted by the methods described herein.

The term “PEG” or “polyethylene glycol” is used to denote an excipient with the general formula of H(OCH₂CH₂)ₙOH which is a linear polymer. The subscript “n” defines the number of ethylene oxide units, which will define its molecular weight (MW) and in this instance can be a range of MWs. Polyethylene glycol can also be generated as “branched PEGs” such as 3-armed, 4-armed, etc. PEGs which can be produced by synthetic means as previously described (Zalipsky and Harris, in Poly[ethlyene glycol] Chemistry and Biological Applications, 1997, p. 8-9, new PEG architectures).

The term “pharmaceutically effective amount” of hGH refers to that amount that provides therapeutic effect in an administration regimen. The compositions hereof are prepared containing amounts of hGH at least about 0.1 mg/ml to about upwards of about 20 mg/ml, preferably from about 1 mg/ml to about 10 mg/ml, more preferably from about 1 mg/ml to about 5 mg/ml. For use of these compositions in administration to human patients suffering from hypopituitary dwarfism, for example, these compositions contain hGH from about 1 mg/ml to about 10 mg/ml, corresponding to the currently contemplated dosage regimen for the intended treatment. In one embodiment, a 5 mg/ml hGH concentration is chosen which allows for an injection volume of 0.27 mL to 0.90 mL based on a dosage of 0.03 to 0.10 mg hGH/Kg body weight (BW)/day and a BW of 45 Kg. As will be obvious to those skilled in the art, the dose of hGH administered to a patient will be determined by the medically accepted dosage for such condition and the dosage regimen.

### General Methods

Unlike lyophilized formulations of hGH as described in U.S. Pat. No. 5,096,885, the current invention does not have a requirement for glycine or any other amino acid typically used for this purpose of a lyoprotectant or cryoprotectant.

The current invention does not have a requirement for Tween or other non-ionic surfactants (i.e., which is comprised of a hydrophilic head and a hydrophobic tail). Additionally, the current invention does not have a requirement for Poloxamer 188 or other ethyleneipropylene block co-polymers.

The suitable pH range for a long-term stable hGH formulation in one embodiment is from 5.5 to 7 and most preferably, pH 6.0. Liquid hGH formulation above pH 7 result in increased protein degradation due to deamidation. As the iso-electric point for recombinant hGH is near pH 5.1, liquid hGH formulations at or near pH 5.1 result in loss of hGH due to aggregation. At a pH considerably less than the iso-electric point, liquid hGH formulations are also not prone to aggregation. In an alternate embodiment, the hGH formulation may have a pH less than 4.5, and more preferably the hGH formulation may have a pH range from 4 to 4.5.

Suitable buffers for the formulations of the present invention are buffers that are pharmaceutically acceptable and provide a buffered pH in the suitable pH range are defined herein. For the pH range 5.5 to 7, suitable buffers include, but are not limited to, phosphate, acetate, citrate, succinate, glycinate, Tris or histidine buffers. The concentration is in the range of 2 to 50 mM.

An effective amount of PEG is added to a pharmaceutical liquid hGH formulation which results in a long term stable pharmaceutical liquid hGH formulation and permits the hGH formulation to be exposed to shear and surface stresses without resulting in significant denaturation of the protein. In a preferred embodiment, the size of the PEG molecules that are added may range from 400-20000 MW, preferably from 1450-10000 MW, more preferably from 3350-8000 MW. The concentration of PEG will range from 5 mg/mL to 100 mg/mL, and preferably 5 mg/mL to 20 mg/mL and more preferably 10 mg/mL to 20 mg/mL. The concentration of PEG required to result in an effective amount is inversely proportional to the molecular weight size of the PEG. Specifically, larger molecular weight PEG molecules are required at a lower concentration.

An antimicrobial agent may also be included in the current formulation. An antimicrobial agent, if included, would retard microbial growth and thereby enable the formulation to be used as a multiple use product. Acceptable antimicrobial agents include 0.2-0.6% (w/v) phenol; 0.7-1% (w/v) benzyl alcohol; m-cresol; methyl-, propyl- or butylparaben or combination of parabens thereof.

A tonicifying agent is added to the formulation and may include a sugar alcohol or sugar, examples of which include, but are not limited to, dextrose, mannitol, glycerol, sorbitol or xylitol; galactose, arabinose or lactose and is added at a concentration required to bring the formulation to be approximately isotonic, i.e., approximately 10 to 100 mg/mL. The present invention does not require the use of tonicity adjusting salts such as NaCl or KCI.

An alternate embodiment of the instant invention is an aqueous human growth hormone formulation comprising human growth hormone, a buffer, a tonicifying agent, a chelating agent and effective amount of Polyethylene glycol, and optionally an antimicrobial agent in a sterile pharmaceutically acceptable liquid. The chelating agent is added to further inhibit potential degradation of the human growth hormone. Chelating agents such as EDTA (ethylenediaminetetraacetic acid) may be used at a concentration of 0.01-0.1%. Other chelating agents known in the art may also be used as noted below.

Additional agents may be added to the formulation and include for example chelating agents such as, preferably, sodium EDTA, calcium disodium EDTA, disodium EDTA, aspartic acid or citric acid, and anti-oxidants such as cysteine, methionine and the like.

### EXPERIMENTAL EXAMPLES

### A. Assay Methods

A₂₈₀ Concentration: The concentration of hGH in the preparations used to make formulations or in non-formulated samples was determined by reading a centrifuged sample at 280 nm in a 1 cm cell and using an extinction coefficient of 0.76 (61 cm, 1 mg/mL, 280 nm=0.76). Typical blank solution was 10 mM Na phosphate, pH 7.4.

SEC Concentration: The concentration of hGH in formulated samples was determined by running standard plots of peak area versus concentration in the range of 1-2 mg/mL. The peak area was determined by running hGH standards on a Superdex 75 HR 10130 column at a flow of 0.9 mL/minute at room temperature and the elution was followed by monitoring at 280 nm. The eluting solvent was 50 mM sodium phosphate, 100 mM sodium sulfate, pH 7.3. Sample loads were 200 μg total protein in a volume of 200 μL.

SEC Purity: The percentage of dimers or aggregates in the formulated samples was determined by performing chromatography as described for SEC concentration. The percentage of dimers or aggregates was determined by dividing the respective peak area by the total peak area.

AEX: The percentage of deamidated hGH in the formulations was determined by running a 200 μg load total protein (in a volume of 100, μL) on a Mono-Q HR 5/5 anion exchange column at a flow of 1.5 mL/minute at room temperature. The eluting solvents were: buffer A=20 mM Tris, pH 8.0 and buffer B=20 mM Tris, 300 NaCl, pH 8.0. The deamidated forms were separated from the native hGH by using a 15 mM NaCl/minute gradient, i.e., 0-60% B in 12 minutes. The eluent was followed at 280 nm. For any formulation that contained an excipient, e.g., phenol, that absorbed at 280, the column was washed with 20 mM Tris, pH 8.0/30% methanol in order to remove the excipient.

RP-HPLC: The percentage of hGH related impurities, i.e., deamidation, oxidation, in the formulations was determined by running a 40 μg load total protein (in a volume of 20 μL) on a Jupiter C4 High pH RP column (5μ, 300 Å, 4.6×250 mm) at a flow rate of 0.5 mL/minute at a temperature of 45° C. Eluent A is 50 mM Tris, pH 8.5 and eluent B is 50% 1-propanol/50 mM Tris, pH 8.5 and the sample is eluted using an isocractic method, where the eluting mixture is 54% eluent B in eluent A. The elution is followed at 220 nm. The percentage of impurities is determined from the following equation:

% impurities=(total peak area−native peak area)/total peak area×100.

LC/MS: The percentage of truncation was estimated by running a 100-200 pmol load total protein on a Michrom PLRP-S column at a flowrate of 0.2 mL/min at a temperature of 35° C. Column eluent is directed into an electrospray ionization probe and molecular mass of the molecular species are determined from the mass spectrum. The percentage truncation is determined from the following equation:

% truncation=(truncation)/total hGH species peak area×100

Antimicrobial testing: Abbreviated antimicrobial testing was performed as per the guidelines set forth in United States Pharmacopoeia USP for formulations set up on long term stability experiments, i.e., the only organism tested was E. coli.

### Formulation Preparation

For the studies pertaining to the physical stability of hGH, solutions were prepared from either lyophilized hGH which was reconstituted and buffer exchanged into 1 mM Na phosphate, pH 6.0 or from bulk liquid hGH containing 10 mM Na phosphate, pH 6.0 and concentrated to an appropriate concentration required for formulation preparation. The concentration of the solutions was determined by reading at A₂₈₀.

For the studies pertaining to the long-term stability (which investigated the physical and chemical stability) of hGH, the formulations were prepared by concentrating bulk hGH solution, pH 6.0, in the presence of either Poloxamer 188 or PEG, and then adding the excipients in the order as indicated. The formulated stock solution was sterile filtered and 1 mL was dispensed into glass vials and hermetically sealed.

In general, the formulations of the subject invention may contain other components in amounts not detracting from the preparation of stable forms and in amounts suitable for effective, safe pharmaceutical administration. For example, other pharmaceutically acceptable excipients well known to those skilled in the art may form a part of the subject compositions. These include, for example, various bulking agents, additional buffering agents, chelating agents, antioxidants, cosolvents and the like.

### Example I

### Physical Stability

It is generally understood in the art of developing aqueous protein pharmaceuticals that an excipient that protects a protein against agitation induced aggregation generally also protects against aggregation induced during long-term storage of such protein formulation. To determine the effect of PEG on the agitation-induced aggregation of hGH, lyophilized hGH was reconstituted and buffer exchanged into 1 mM sodium phosphate, pH 6.0. Solutions were prepared with PEG-3400 added to final concentrations of 2.0, 5.0, 10.0 and 20 mg/mL, and the final hGH concentration being 0.5 mg/mL in a total of 1.5 mL. Negative control solutions (i.e., containing no PEG or poloxamer 188) and positive controls (i.e., containing 1 mg/mL Poloxamer 188) were included in the sample sets as reference. Triplicate samples of each solution were vigorously vortexed in glass test tubes for 5 minutes and then allowed to incubate at room temperature for 30 minutes. The samples are then centrifuged to clear aggregates of hGH that are formed. Following centrifugation, the percent hGH loss for triplicate samples was then determined by reading the absorbance at A₂₈₀.

TABLE I
Vortex study using 0.5 mg/mL hGH in 1 mM Na phosphate, pH 6.0
and varying concentrations of PEG-3400

| | % hGH loss by |
|---|---|
| Sample | A₂₈₀ (avg of 3) |
| Negative Control (no Poloxamer 188 or PEG) | 36 |
| Positive control (1 mg/mL Poloxamer 188) | 3 |
| PEG-3400, 2.0 mg/mL | 34 |
| PEG-3400, 5.0 mg/mL | 27 |
| PEG-3400, 10.0 mg/mL | 25 |
| PEG-3400, 20.0 mg/mL | 16 |

The results summarized in Table I demonstrate that aqueous hGH samples without Poloxamer 188 or PEG result in a loss of 36% of the hGH. In contrast, in the presence of Poloxamer 188, there is only a 3% loss of hGH. Thus, Poloxamer 188 decreases the aggregation of hGH by 12 fold. Agitation of the solution containing PEG results only in a 0 to 2 fold decease in loss of hGH (i.e. from 34% to 16%). From the above results, it appears that PEG offers minimal protection to hGH against agitation induced aggregation (i.e. 6 fold less protection that Poloxamer 188), suggesting PEG will not provide long term stability to an hGH liquid formulation. As described in the Background of the instant Specification, previous studies have demonstrated PEG destabilizes certain proteins. From the above results, it appears that PEG provides less stabilization to hGH compared to Poloxamer.

Additional agitation-induced aggregation experiments were performed with PEG molecules of various molecular weight (MW) sizes. Lyophilized hGH was buffer exchanged into 1 mM sodium phosphate, pH 6.0 and either 10.0 or 20 mg/mL PEG of differing MW was added, i.e., MW of 400, 600, 1000, 1450, 3400, 6000, 8000, 12000 or 20000, with the final hGH concentration being 0.5 mg/mL in a total of 1.25 mL. Negative controls (i.e., containing no PEG or poloxamer 188) and positive controls (i.e., containing 2 mg/mL Poloxamer 188) were included in the sample sets as reference. As previously performed, the solutions were vigorously vortexed in glass test tubes for 5 minutes and then allowed to incubate at room temperature for 30 minutes. The percent hGH loss for the samples was then determined by centrifuging and reading at A₂₈₀.

TABLE II
Vortex study using 0.5 mg/mL hGH in 1 mM Na phosphate, pH 6.0
and varying MWs of PEG containing either 10 or 20 mg/mL PEG

| | | % hGH loss |
|---|---|---|
| Sample | | by A₂₈₀ |
| Negative Control (no | | 58 |
| Poloxamer 188 or PEG) | | |
| Positive control | | 7 |
| (2 mg/mL Poloxamer 188) | | |
| PEG-400 | 10 mg/mL | 37 |
| | 20 mg/mL | 38 |
| PEG-600 | 10 mg/mL | 26 |
| | 20 mg/mL | 30 |
| PEG-1000 | 10 mg/mL | 45 |
| | 20 mg/mL | 38 |
| PEG-1450 | 10 mg/mL | 24 |
| | 20 mg/mL | 28 |
| PEG-3400 | 10 mg/mL | 19 |
| | 20 mg/mL | 18 |
| PEG-6000 | 10 mg/mL | 17 |
| | 20 mg/mL | 17 |
| PEG-8000 | 10 mg/mL | 16 |
| | 20 mg/mL | 16 |
| PEG-12000 | 10 mg/mL | 16 |
| | 20 mg/mL | 16 |
| PEG-20000 | 10 mg/mL | 16 |
| | 20 mg/mL | 14 |

The results documented in Table II demonstrate that samples without Poloxamer 188 or PEG result in 58% of the hGH being lost. The presence of Poloxamer 188 results in only a 7% loss of hGH. The addition of low molecular weight PEG (PEG-400 or PEG-600) results in a 34% loss of hGH. There appears to be a plateau in the protective effect of PEG molecules above PEG-3400, with loss of hGH about 16%. There does not appear to be a difference in the protective effect for PEG concentration of either 10 mg/mL or 20 mg/mL. However, as the previous results documented in Table 1 have demonstrated, PEG does not offer the same level of protection against agitation induced aggregation as Poloxamer 188. Specifically, Poloxamer 188 offers an 8 fold decrease in aggregation, while PEG offers only a 3 fold decrease in aggregation.

The results from the agitation induced aggregation experiments summarized in Tables I and II suggest that PEG will offer only minimal protection to hGH for long term stability. Specifically, the results suggest that PEG offers about 3 to 6 fold less protection from aggregation compared to Poloxamer 188.

Despite the results of the previous experiments, a pharmaceutical formulation was prepared composed of 5 mg/mL hGH, 3.2 mg/mL phenol, 7.5 mg/mL NaCl, 10 mg/mL PEG3400 and 10 mM Na phosphate pH 6.0. However, a precipitate formed in such formulation due to the presence of NaCl together with PEG (data not shown).

However, in a surprising and unexpected result, it was discovered that in formulations containing hGH at concentrations above 1 mg/ml, and in the presence of a buffer and a sugar or sugar alcohol tonicifying agent, the PEG formulations protected hGH against agitation induced aggregation as well as, and in some cases, better than the Poloxamer 188 formulation. An exemplary example is provided below. Formulations were prepared in triplicate comprising 5 mg/mL hGH, 10 mM sodium phosphate, pH 6.0, 43 mg/mL dextrose (a tonicity agent) and 3.2 mg/mL phenol (an antimicrobial that is not required for protection against agitation induced aggregation) in a final volume of 1 mL. As previously described, the formulations were vigorously vortexed in glass test tubes for 5 minutes and then allowed to incubate at room temperature for 30 minutes. Following certrifugation, the percent hGH loss for the samples was then determined by the SEC concentration assay. The percent increase in dimer and aggregate formation was also determined from the SEC purity assay.

TABLE III
Vortex study using 5 mg/mL hGH in 10 mM Na phosphate, pH 6.0,
10 mg/mL PEG-3400 or -8000 (or 2 mg/ml Poloxamer 188),
43 mg/mL dextrose and 3.2 mg/mL phenol

| | % hGH | % Increase in | % Increase |
|---|---|---|---|
| Sample | loss by SEC | Dimer | in Aggregate |
| Negative Control | 7.3 | 30 | 16 |
| (no Poloxamer 188 or PEG) | | | |
| Positive Control | 1.3 | 6.3 | 5.8 |
| (2 mg/mL Poloxamer 188) | | | |
| PEG-3400, 10 mg/mL | −0.2 | 4.0 | 1.3 |
| PEG-8000, 10 mg/mL | −0.4 | 0.0 | −3.6 |

The negative control formulation without Poloxamer 188 or PEG results in 7.3% loss of hGH and a significant increase in dimer and aggregate formation, i.e., 30% and 16% respectively. Formulations containing Poloxamer 188 result in a 1.3% loss of hGH and a reduction in the level of dimer and aggregate formation compared to the negative control. Surprisingly, for formulations containing PEG, there is essentially no loss in hGH. The PEG-3400 formulation resulted in less dimer and aggregate than observed in the Poloxamer 188 formulation. Even more surprisingly, the PEG-8000 formulation shows no formation of dimers and an apparent reduction in the level of aggregates. This apparent reduction in aggregate formation may result from the dissolution of hGH aggregates present in the starting solution. The results demonstrate that in the presence of buffer and a tonicifying agent, PEG formulated hGH at concentration above 1 mg/ml is as stable, and perhaps more stable against agitation-induced aggregation than Poloxamer 188 formulations.

### Example II

### Long Term Stability (Including Examination of Chemical and Physical Stability)

The long term stability of hGH was compared for formulations containing PEG or poloxamer 188. The PEG formulation contained: 5 mg/mL hGH, 10 mM Na phosphate, pH 6.0, 47 mg/mL mannitol, 10 mg/mL PEG-3400 and 3.2 mg/mL phenol. A control formulation, the poloxamer 188 formulation, contained: 5 mg/mL hGH, 10 mM Na phosphate, pH 6.0, 7.5 mg/mL NaCl, 2 mg/mL Poloxamer 188 and 3.2 mg/mL phenol. The formulations were incubated at either the recommended storage temperature of 2 to 8° C. or at an elevated storage temperature of 25° C. Samples were removed at various time points and the hGH was analyzed for physical and chemical stability as described above, i.e., by SEC, for loss of hGH, percent increase in dimer and aggregate and as well as, by RP-HPLC and AEX (to follow chemical degradation).

TABLE IV
Stability Study of a Poloxamer 188 versus a PEG Formulation
PEG formulation: 5 mg/mL hGH, 3.2 mg/mL phenol, 47 mg/mL mannitol,
10 mg/mL PEG-3400, 10 mM Na phosphate pH 6.0
Poloxamer 188 formulation: 5 mg/mL hGH, 3.2 mg/mL phenol, 7.5 mg/mL
NaCl, 2 mg/mL poloxamer 188, 10 mM Na phosphate pH 6.0
Incubation at 2-8° C.:

| | | | | | | % Increase (decrease) | | |
|---|---|---|---|---|---|---|---|---|
| | | Assay result | | | | from time zero | | |
| Formulation | Test | Time zero | 2 weeks | 1 month | 3 month | 2 weeks | 1 month | 3 month |
| PEG | Conc'n | 5.1 | 5.0 | 5.1 | 5.6 | (2.0) | 0 | 10 |
| Poloxamer | | 5.6 | 5.5 | 5.5 | 5.9 | (1.8) | (1.8) | 5 |
| 188 | | | | | | | | |
| PEG | % Dimer | 0.4 | 0.5 | 0.5 | 1.0 | 25 | 25 | 150 |
| Poloxamer | | 0.4 | 0.5 | 0.5 | 1.0 | 25 | 25 | 150 |
| 188 | | | | | | | | |
| PEG | % Aggregates | 0.6 | 0.7 | 0.7 | 1.2 | 17 | 17 | 100 |
| Poloxamer | | 0.6 | 0.7 | 0.6 | 1.3 | 17 | 0 | 117 |
| 188 | | | | | | | | |
| PEG | % | 3.6 | 4.2 | 3.7 | 6.7 | 17 | 2.8 | 86 |
| Poloxamer | Deamidation | 3.4 | 4.5 | 4.0 | 9.0 | 32 | 18 | 165 |
| 188 | | | | | | | | |
| PEG | % RP | 16.0 | 16.2 | 15.9 | 20.1 | 1.3 | 0.6 | 26 |
| Poloxamer | impurities | 14.5 | 15.9 | 16.6 | 22.7 | 9.7 | 15 | 57 |
| 188 | | | | | | | | |

Incubation at 25° C.:

| | | % Increase (decrease) | | | | |
|---|---|---|---|---|---|---|
| | | from time zero | | | | |
| Formulation | Test | Time zero | 2 weeks | 1 month | 2 weeks | 1 month |
| PEG | Conc'n | 5.1 | 5.1 | 4.9 | 0 | (3.9) |
| Poloxamer | | 5.6 | 5.4 | 5.4 | (3.6) | (3.6) |
| 188 | | | | | | |
| PEG | % Dimer | 0.4 | 0.6 | 0.8 | 50 | 100 |
| Poloxamer | | 0.4 | 0.7 | 1.0 | 75 | 150 |
| 188 | | | | | | |
| PEG | % Aggregates | 0.6 | 0.7 | 0.9 | 17 | 50 |
| Poloxamer | | 0.6 | 0.8 | 1.0 | 25 | 67 |
| 188 | | | | | | |
| PEG | % | 3.6 | 8.0 | 12.7 | 122 | 253 |
| Poloxamer | Deamidation | 3.4 | 8.9 | 15.0 | 162 | 341 |
| 188 | | | | | | |
| PEG | % RP | 16.0 | 21.5 | 26.7 | 34 | 67 |
| Poloxamer | impurities | 14.5 | 22.5 | 28.7 | 55 | 98 |
| 188 | | | | | | |

Given the results in table III above, we expected the rates of hGH aggregation following long term storage would be comparable between hGH formulated in either PEG or Poloxamer 188. However, surprisingly, the storage stability testing demonstrated a decreased rate of chemical degradation of hGH formulated in the PEG formulation as compared to the Poloxamer 188 formulation. As expected, the variations observed at one month in the sample stored at 2 to 8° C. are small. Because the variations are small, they are likely due to inaccuracies inherent in assays at the lower level of detection. As expected, both physical and chemical degradation were accelerated in the samples stored at 25° C. With respect to physical degradation of hGH, after one month of storage, 33% fewer hGH dimers and aggregates were detected in the PEG formulation compared to the Poloxamer 188 formulation. Similarly, 33% lower levels of chemically degraded hGH (deaminated hGH and RP impurities) were detected in the PEG formulation as compared to the Poloxamer 188 formulations. The above results demonstrate a formulation comprising PEG, a buffer, a tonicifying agent and having hGH concentrations above 1 mg/ml protects hGH against both physical and chemical degradation during long term storage. Generating stability data at the recommended storage conditions would require incubation at 2 to 8° C. for 12-24 months. As such, accelerated stability studies, performed at elevated temperatures, may be used as an indication of the stability of the product at the normal storage conditions (Chang B. S. and Hershenson S. Pharm Biotechnol. 2002;13:1-25).

Predicting Long Term Stability of the Liquid Formulation from 1 Month Data.

The long term stability of hGH formulated in the PEG formulation was predicted from the accelerated stability studies at 25° C., with respect to chemical and physical degradation of the hGH molecule. To increase the accuracy of the prediction, data from a 24 month stability study of hGH formulated in Poloxamer 188 was used as a reference (the “24 month study”). Such 24 month stability study is based on the formulation: 5.0 mg/mL hGH, 10 mM sodium phosphate, pH 6.0, 2.0 mg/mL poloxamer 188 and 2.5 mg/mL phenol with stability data was collected at 5 and 25° C. The information obtained from this stability study was used to calculate the activation energy (Ea).

To compare the above described hGH formulated in the PEG formulation with the of hGH formulated in the Poloxamer 188 formulation with respect to the level of deamidation, rate constants were determined for each formulation at 25° C. followed by the rate constants at 5° C., using the Arrhenius equation. Subsequently, the rate data at 5° C. was then used to determine the percentage of the deamidated forms of hGH at 6, 12, 18 and 24 months. The following table summarizes the information obtained.

TABLE V
Predicted Percentage of Deamidated Forms of hGH

| | | | | | % | | |
|---|---|---|---|---|---|---|---|
| | | Rate | % Deamid | % Deamid | Deamid | % Deamid | Eₐ (Joule/ |
| Formulation | Temp | (day⁻¹) | (6 M) | (12 M) | (18 M) | (24 M) | mole) |
| 24 month | 5° C. | 1.5 × 10⁻⁴⁽¹⁾ | NA | NA | NA | 10.3%⁽²⁾ | 95,376⁽³⁾ |
| stability | 25° C. | 0.0024⁽¹⁾ | | | | NA | |
| PEG | 5° C. | 2.0 × 10⁻⁴⁽⁵⁾ | 3.6%⁽⁶⁾ | 7.0%⁽⁶⁾ | 10.2%⁽⁶⁾ | 13.8%⁽⁶⁾ | NA |
| Formulation | 25° C. | 0.0032⁽⁴⁾ | NA | NA | NA | NA | |
| Poloxamer | 5° C. | 2.6 × 10⁻⁴⁽⁵⁾ | 4.4%⁽⁶⁾ | 8.8%⁽⁶⁾ | 13.2%⁽⁶⁾ | 17.2%⁽⁶⁾ | |
| 188 | 25° C. | 0.0042⁽⁴⁾ | NA | NA | NA | NA | |
| Formulation | | | | | | | |

⁽¹⁾Determined from 24 month data.
⁽²⁾Actual percent deamidation at 24 months.
⁽³⁾Calculated using Arrhenius equation.
⁽⁴⁾Calculated from 1 month data.
⁽⁵⁾Predicted from Arrhenius equation.
⁽⁶⁾Extrapolated

The rate constants for each of the current PEG and current Poloxamer 188 formulations at 25° C. determined from the one month data, and are on the same order as the 25° C. data for the 24 month study, suggesting that the rate constants determined at 5° C. from the 24 month study will give a reasonable estimate of the percent deamidation at 6, 12, 18 and 24 month time point for the PEG and Poloxamer 188 formulations. From the table, it can be see that the PEG formulation will provide better protection against deamidation than the Poloxamer 188 formulation.

To compare the current PEG formulation with the control Poloxamer 188 formulation with respect to the level of aggregation, rate constants were determined (based on the principle set out in Kendrick et. al., 1998) for each formulation at 25° C. followed by the rate constants at 5° C., using the Arrhenius equation. Subsequently, the rate data at 5° C. was then used to determine the percentage of the aggregated forms of hGH at 24 months. The following table summarizes the information obtained.

TABLE VI
Predicted Percentage of Aggregated Forms of hGH at 24 Months

| | | Rate | % Aggre | % Aggre | % Aggre | % Aggre | Eₐ (Joule/ |
|---|---|---|---|---|---|---|---|
| Formulation | Temp | (day⁻¹) | (6 M) | (12 M) | (18 M) | (24 M) | mole) |
| 24 month | 25° C. | 8.5 × 10⁻⁵⁽¹⁾ | NA | NA | NA | 0.51%⁽²⁾ | 125,683⁽³⁾ |
| stability | 37° C. | 6.07 × 10⁻⁴⁽¹⁾ | | | | NA | |
| PEG | 5° C. | 2.667 × 10⁻⁶⁽⁵⁾ | 0.04%⁽⁶⁾ | 0.08%⁽⁶⁾ | 0.12%⁽⁶⁾ | 0.18%⁽⁶⁾ | NA |
| Formulation | 25° C. | 0.000103⁽⁴⁾ | NA | NA | NA | NA | |
| Poloxamer | 5° C. | 3.522 × 10⁻⁶⁽⁵⁾ | 0.04%⁽⁶⁾ | 0.12%⁽⁶⁾ | 0.18⁽⁶⁾ | 0.26%⁽⁶⁾ | |
| 188 | 25° C. | 0.000136⁽⁴⁾ | NA | NA | NA | NA | |
| Formulation | | | | | | | |

⁽¹⁾Determined from 24 month data.
⁽²⁾Actual percent aggregation at 24 months.
⁽³⁾Calculated using Arrhenius equation.
⁽⁴⁾Calculated from 1 month data.
⁽⁵⁾Predicted from Arrhenius equation.
⁽⁶⁾Extrapolated

The rate constants for each of the PEG and Poloxamer 188 formulations at 25° C. determined from the one month data is on the same order as the 25° C. data for the ancillary poloxamer 188 study, suggesting that the rate constants determined at 5° C. will give a reasonable estimate of the percent deamidation at the 24 month time point for the PEG and Poloxamer 188 formulations. The PEG formulation prevents aggregation of hGH on the same order as the Poloxamer 188 formulation and thus is comparable to a Polxoamer formulation.

Based on the physical and chemical degradation rates of hGH observed in the sample incubated at 25° C., an extrapolation was made to demonstrate long-term storage stability of hGH in the PEG formulation at least 6 months and up to 18 months. Extrapolation of the aggregation rate and the deamidation rate demonstrate that hGH formulated in the PEG formulation will have less than 3-4% aggregation and less that 15% deamidation. Such rates are acceptable and will result in an hGH pharmaceutical formulation that can be administered to human patients in need thereof.

### Example III

### Preservative Effectiveness

To test the antimicrobial effectiveness of the PEG of Poloxamer 188 formulations, an abbreviated procedure of the USP was used, i.e., testing was done with E. coli only. Briefly, six vials of each formulation were combined, under sterile conditions, into one aliquot of a volume of 6 mL. A qualified inoculum of the bacterium was then added to the formulation at a final concentration of 1×10⁵ to 1×10⁶cfu/mL and allowed to incubate at 37° C. for the following time periods: 7 days, 14 days and 1 month. At each time period, sample is withdrawn and the numbers of cfu are determined. The required kill for a category 1A product is not less than 1 log reduction at 7 days, not less than a 3 log reduction at 14 days and no increase from day 14 at 28 days. As such, the antimicrobial effectiveness of the formulation was achieved and the E. coli kill for the PEG formulation is comparable to the Poloxamer formulation.

TABLE VII
Antimicrobial Effectiveness for PEG and Poloxamer 188
Formulations

| | Log, Initial | Log of Count | | | Log Reduction | | |
|---|---|---|---|---|---|---|---|
| Sample | Count/* | 7 Days | 14 Days | 1 Month | 7 Days | 14 Days | 1 Month |
| PEG | 5.61 | 2.57 | <1.00 | <1.00 | 3.04 | >4.61 | >4.61 |
| Poloxamer | 5.61 | 1.0 | <1.00 | <1.00 | 4.61 | >4.61 | >4.61 |
| 188 | | | | | | | |

### Example VI

### Chelating Agent

The inventors also discovered that the addition of a chelating agent to the aqueous human growth hormone formulation further inhibited degradation of the human growth hormone. Formulations containing hGH, mannitol, PEG-3400, Na phosphate and phenol at concentrations described above were mixed with various concentration of EDTA. The formulations were incubated at 37° C. in an accelerated stability study. At 2 weeks, 1 and 2 month time points, samples were then analyzed by various methods including reversed phase HPLC (RP) and the percent increase in degraded/truncated hGH products were quantitated. As detailed in Table VIII, increased EDTA concentrations appear to slow the rate of hGH degradation at all time points analyzed.

TABLE VIII
Effect of EDTA on hGH stability indicative parameters

| EDTA | % | | % RP Impurities (Deamidated + | | | |
|---|---|---|---|---|---|---|
| Conc'n | Truncated at 37° C. | | Truncated + Oxidized) at 37° C. | | | |
| (mg/mL) | T = 0 | 2 weeks | T = 0 | 2 weeks | 1 month | 2 months |
| 0 | 0.0 | 6.1 | 5.7 | 30.3 | 46.1 | 69.4 |
| 0.1 | 0.0 | 5.5 | 5.9 | 28.3 | 43.4 | 66.5 |
| 0.4 | 0.0 | 4.0 | 6.2 | 26.1 | 39.7 | 62.3 |
| 0.7 | 0.0 | 4.3 | 6.5 | 24.2 | 36.9 | 60.4 |
| 1.0 | 0.0 | 4.4 | 5.8 | 21.9 | 34.6 | 56.2 |

Unlike the 25° C. accelerated stability study summarized in Tables V and VI herein above, incubation at 37° C. described in this Example VI is not an approximate of stability storage at 2 to 8° C. Incubation at 37° C. is far harsher than incubation at 25° C. and is only meant to test if a chelating agent inhibits hGH degradation.

Preparing hGH formulations of the alternate embodiment of the invention with EDTA may require some attention to the final pH of the formulation to avoid the formation of a precipitate. The addition of EDTA decreases the pH of the formulation. An hGH formulation of the instant invention with EDTA results in the formation of a precipitate at pH levels at or below pH 6.0. To inhibit the formation of such precipitate, the pH of the Na phosphate buffer used to formulate the hGH is increased to pH 6.7, such that the final hGH formulation is about pH 6.4. One example of an embodiment of the invention is an aqueous hGH formulation having a pH of about 6.4 comprising:

1 mg/mL to 20 mg/mL hGH

a tonicifying agent

5 mg/mL to 20 mg/mL PEG

buffer system providing about pH 6.4

0.1 mg/mL to 1 mg/ml of a chelating agent

and optionally an antimicrobial agent.

More specifically, there is provided an aqueous hGH formulation having a pH of about 6.4 comprising:

5 mg/mL hGH

about 50 mg/mL mannitol

15 mg/mL PEG-3400

10 mM Na phosphate

1.9 mM EDTA

and optionally 2 mg/ml to 6 mg/mL phenol.

While the preferred embodiments of the invention have been described above, it will be recognized and understood that various modifications may be made therein, and the appended claims are intended to cover all such modifications which may fall within the spirit and scope of the invention.

1. A human growth hormone formulation comprising:
a) 1 mg/ml to 20 mg/ml human growth hormone,
b) buffer system providing pH 5.5 to pH 7,
c) a tonicifying agent, and
d) an effective amount of Polyethylene glycol,
in a sterile pharmaceutically acceptable liquid.

2. The human growth hormone formulation of claim 1 wherein the polyethylene glycol is PEG 1450 to PEG 20000.

3. The human growth hormone formulation of claim 2 wherein the polyethylene glycol is 5 mg/mL to 50 mg/mL.

4. The human growth hormone formulation of claims 1 wherein said formulation is long term cold storage stable for 6 to 18 months at 2° C. to 8° C.

5. The human growth hormone formulation of claim 4 including an antimicrobial agent.

6. The human growth hormone formulation of claim 5 wherein the polyethylene glycol is PEG 1450 to PEG 20000.

7. The human growth hormone formulation of claim 6 wherein the polyethylene glycol is 5 mg/mL to 50 mg/mL.

8. The human growth hormone formulation of claim 4 wherein the buffer system provides a pH 6.

9. The human growth hormone formulation of claim 8 wherein the tonicifying agent is mannitol.

10. The human growth hormone formulation of claim 4 including a chelating agent.

11. The human growth hormone formulation of claim 10 wherein the buffer system provides about pH 6.4.

12. The human growth hormone formulation of claim 11 including an antimicrobial agent.

13. A method for using human growth hormone comprising the steps of
A) formulating said human growth hormone into an aqueous liquid formulation comprising:
a) 1 mg/ml to 20 mg/ml human growth hormone,
b) buffer system providing pH 5.5 to pH 7,
c) 5 mg/mL to 50 mg/mL polyethylene glycol, and
d) a tonicifying agent,
in a pharmaceutically acceptable, injectable sterile aqueous vehicle,
B) storing said formulation as an aqueous liquid for from six to 18 months at 2° C. to 8° C. thereby forming a stored formulation; and
C) directly injecting said stored formulation into a patient in need of human growth hormone therapy.

14. A method for using human growth hormone comprising the steps of
A) formulating said human growth hormone into an aqueous liquid formulation consisting essentially of:
a) 1 mg/ml to 20 mg/ml human growth hormone,
b) buffer system providing pH 5.5 to pH 7,
c) 5 mg/mL to 50 mg/mL polyethylene glycol,
d) 20 to 100 mg/mL of a tonicifying agent and
e) an antimicrobial agent,
in a pharmaceutically acceptable, injectable sterile aqueous vehicle,
B) storing said formulation as an aqueous liquid for from six to 18 months at 2° C. to 8° C. thereby forming a stored formulation; and
C) directly injecting said stored formulation into a patient in need of human growth hormone therapy.

15. A method of making a storage stable aqueous formulation of human growth hormone comprising mixing said human growth hormone into an aqueous, pharmaceutically acceptable vehicle which comprises
a) 1 mg/ml to 20 mg/ml of said human growth hormone;
b) buffer providing pH 5.5 to pH 7;
c) 5 mg/mL to 50 mg/mL polyethylene glycol; and
d) 20 to 100 mg/mL of a tonicifying agent;
wherein said aqueous, pharmaceutically acceptable vehicle is capable of storage for 6 to 18 months at 2 to 8° C.
